(19) United States
(12) Patent Application Publication
(10) Pub. No.: US 2023/0080670 A1
(43) Pub. Date: Mar. 16, 2023

(54) METHODS AND COMPOSITIONS FOR TREATING TUMORS USING TRANSCRIPTION INHIBITION AND DNA DAMAGE

(71) Applicant: ARIZONA BOARD OF REGENTS ON BEHALF OF THE UNIVERSITY OF ARIZONA, Tucson, AZ (US)

(72) Inventors: Jacob Schwartz, Tucson, AZ (US); Matthew Rollins, Tucson, AZ (US); Nasiha S. Ahmed, Tucson, AZ (US)

(21) Appl. No.: 17/600,817

(22) PCT Filed: Apr. 6, 2020
(86) PCT No.: PCT/US20/26926

§ 371 Date: Oct. 1, 2021

Related U.S. Application Data
(60) Provisional application No. 62/830,278, filed on Apr. 5, 2019.

(51) Int. Cl. A61K 31/4745; A61K 31/502; A61K 31/4184; A61K 31/453

(52) U.S. Cl. CPC A61K 31/4745; A61K 31/502; A61K 31/4184; A61K 31/453

(57) ABSTRACT

Methods and compositions for inducing cell death in cells with FET-fused oncogenes, as well as methods for treating tumors with FET-fused oncogenes, such as cells and tumors associated with Ewing's sarcoma and fibromyxoid Iposarcoma. Oncogenes may include the EWS-FLI1 oncogene, FUS-FLI1 oncogene, FUS-CHOP oncogene, etc. The methods feature herein administering to a patient with a FET-fused oncogene tumor a combination of a DNA damaging agent and a transcription inhibitor. The combination of the transcription inhibitor and DNA damaging agent causes death of cells in the tumor.

### CROSS-REFERENCES TO RELATED APPLICATIONS

This application claims benefit of U.S. Patent Application No. 62/830,278 filed Apr. 5, 2019, the specification(s) of which is/are incorporated herein in their entirety by reference.

### STATEMENT REGARDING FEDERALLY SPONSORED RESEARCH OR DEVELOPMENT

This invention was made with government support under Grant No. R21 CA238499 awarded by the National Institutes of Health. The government has certain rights in the invention.

### BACKGROUND OF THE INVENTION

### Field of the Invention

The present invention relates to treatments for tumors such as those associated with FET-fused oncogenes, such as but not limited to Ewing's sarcoma and fibromyxoid liposarcoma, more particularly to the use of a combination of transcription inhibitors and DNA damaging agents for treatment.

### Background Art

Tumors typically arise from diverse and complex genetic backgrounds. Most often, multiple genetic abnormalities are required for transformation and tumorigenesis, which in turn tend to promote genome instability such that tumor cells acquire further mutations as they grow and divide. The heterogeneity of genetic landscapes may limit the effectiveness of biochemical models of tumorigenesis. In contrast, tumors resulting from translocation events involved the FET family of factors (FUS, EWSR1, and TAF15) make up a well-defined and relatively straightforward genetic pathology. An example would be Ewing's sarcoma, which is an aggressive primary bone tumor affecting 1 in 300,000 people annually. More than 80% of Ewing's sarcoma tumors occur in adolescents, making it the second most common pediatric bone cancer.

Eighty-five percent of Ewing's sarcomas are caused by translocations at loci containing the genes EWS and FLI1, which creates a powerful oncogene—EWS-FLI1. Another 10% of cases are caused by translocations between an EWS homologue, FUS, and FLI1. These tumors are typified by a “quiet genome” with only few secondary mutations found in patient samples. Half of known translocations identified in sarcomas involve one of a family of three proteins—FUS, EWS, and TAF15, known as the FET family of proteins. These FET-fusion proteins frequently occur with ETS-family transcription factors (e.g. FLI1, ERG, DDIT3) but can involve other factors (e.g. CREB3L2, ATFI).

The wild-type proteins, FUS and EWS, from which oncogenic fusions are derived, both regulate transcription and are required for DNA damage repair. A knockout of either results in severe chromatin instability. It was surprisingly discovered that FUS fusion proteins and EWS fusion proteins have the effect of leaving their sarcomas, such as Ewing's sarcoma, susceptible to DNA damage.

### BRIEF SUMMARY OF THE INVENTION

The present invention features methods and compositions for treating tumors involving translocation events that create FET-fusion proteins, such as but not limited to Ewing's sarcoma. It was surprisingly discovered that a standard treatment for Ewing Sarcoma using DNA damaging agents is far more effective when combined with a transcription inhibitor. The methods and compositions feature the administration of a transcription inhibitor in combination with a DNA damaging agent for tumors having a fusion oncogene involving a FET protein.

The present invention features methods for inhibiting growth of, inhibiting replication of, or inducing cell death in a cell having a FET-fused oncogene. In certain embodiments, the method comprises introducing to the cell having the FET-fused oncogene an effective amount of a composition comprising a transcription inhibitor and a DNA damaging agent. The composition inhibits growth of the cell, inhibits replication of the cell, or induces cell death in the cell.

The presence of a FET-fused oncogene gives rise to a synergistic effect between inhibition of transcription and DNA damage. Under these conditions, the addition of a transcription inhibitor can lead to greater effectiveness for DNA damage treatments. Similarly, the FUS-fused protein will allow transcription inhibitors to provide a therapeutic effect at doses far lower when combined with the DNA damage treatment than alone.

The method of leveraging the synergy between transcription inhibitors and DNA damage agents in tumors having the FET-fusion protein can allow a therapeutic effect at lower doses or subdose of the agents alone. The presence of the FET-fusion protein can be used as a biomarker of tumors that can be exceptionally sensitive to a combination treatment using transcription inhibiting agents and DNA damaging therapies. Therapeutic doses of transcription inhibitors (such as the CDK and transcription inhibitor flavopiridol) has shown significant toxicity in patients, which has prevented its clinical use for most tumors to enter trials to date. Combination treatments of DNA damage treatments and the maximum tolerated dose flavopiridol can enhance therapeutic benefits but as yet has failed to reach clinical significance (Ang et al., Gastrointest Cancer Res 2012; Cicenas et al., Cancers (Basel) 2014). Illustrating the surprising findings of the synergistic effect between inhibition of transcription and DNA damage, Inventors have found that tumor cells possessing the FET-fusion protein cannot grow or survive at doses of flavopiridol and the DNA damage agent irinotecan three to ten times (3-10 times) lower than those doses that have been used to achieve therapeutic effect in clinical trials (Ducreux et al., Ann Oncol 2003; Shah et al., Clin Cancer Res 2005; Deep et al., New J Chem 2018).

The cell may be a cell of a tumor. Examples of such tumors include Ewing's sarcoma, desmoplastic small round cell tumor, myxoid liposarcoma, clear cell sarcoma, extraskeletal myxoid chondrosarcoma, fibromyxoid sarcoma, and those listed in Table 1. In certain embodiments, the cell having the FET-fused oncogene is a cell of a Ewing's sarcoma tumor. In certain embodiments, the cell having the FET-fused oncogene is a cell of a fibromyxoid liposarcoma tumor. In certain embodiments, the FET-fused oncogene is EWS-FLI1. In certain embodiments, the FET-fused oncogene is FUS-FLI1. In certain embodiments, the FET-fused oncogene is FUS-CHOP. In certain embodiments, the DNA damaging agent is a topoisomerase poison. In certain embodiments, the DNA damaging agent is a DNA crosslinker. In some embodiments, the DNA damaging agent is a DNA repair inhibitor. In certain embodiments, the DNA damaging agent is an ionizing radiation therapy. In certain embodiments, the DNA damaging agent targeted radiation therapy. In certain embodiments, the DNA damaging agent is a topoisomerase poison, a DNA crosslinker, ionizing radiation therapy, or targeted radiation therapy.

For any of the embodiments herein, in certain embodiments, the topoisomerase poison may be one or a combination of is irinotecan, topotecan, camptothecin, diflomotecan, gimatecan, doxorubicin, etoposide, mitoxantrone, or daunorubicin, or the like. In certain embodiments, the topoisomerase poison is irinotecan. In certain embodiments, the topoisomerase poison is topotecan. In certain embodiments, the topoisomerase poison is camptothecin. In certain embodiments, the topoisomerase poison is diflomotecan. In certain embodiments, the topoisomerase poison is gimatecan. In certain embodiments, the topoisomerase poison is doxorubicin. In certain embodiments, the topoisomerase poison is etoposide. In certain embodiments, the topoisomerase poison is mitoxantrone. In certain embodiments, the topoisomerase poison is daunorubicin. In certain embodiments, the DNA crosslinker is one or a combination of cisplatin, carboplatin, or oxaliplatin, or the like. In certain embodiments, the DNA crosslinker is cisplatin. In certain embodiments, the DNA crosslinker is carboplatin. In certain embodiments, the DNA crosslinker is oxaliplatin. In certain embodiments, the DNA repair inhibitor is one or a combination of olaparib, veliparib, CD00509, KU-55933, vorinostat, valproic acid, or VE-821, or the like. In certain embodiments, the DNA repair inhibitor is olaparib. In certain embodiments, the DNA repair inhibitor is veliparib. In certain embodiments, the DNA repair inhibitor is CD00509. In certain embodiments, the DNA repair inhibitor is KU-55933. In certain embodiments, the DNA repair inhibitor is vorinostat. In certain embodiments, the DNA repair inhibitor is valproic acid. In certain embodiments, the DNA repair inhibitor is VE-821.

For any of the embodiments herein, in certain embodiments, the transcription inhibitor is an RNA Pol II targeting kinase inhibitor. In certain embodiments, the transcription inhibitor is a cyclin-dependent kinase inhibitor. In certain embodiments, the transcription inhibitor is a DNA/RNA blocker. In certain embodiments, the transcription inhibitor is a cyclin-dependent kinase inhibitor or a DNA/RNA blocker. In certain embodiments, the transcription inhibitor is a cyclin-dependent kinase inhibitor selected from: flavopiridol, DRB, binaciclib, roscovitine, olomoucine II, or TG02, a combination thereof, or the like. In certain embodiments, the transcription inhibitor is flavopiridol. In certain embodiments, the transcription inhibitor is DRB. In certain embodiments, the transcription inhibitor is binaciclib. In certain embodiments, the transcription inhibitor is roscovitine. In certain embodiments, the transcription inhibitor is olomoucine II. In certain embodiments, the transcription inhibitor is TG02. In certain embodiments, the DNA/RNA blocker is one or a combination of alpha-Amanitin, actinomycin D, cordycepin, fludarabine, or ethidium bromide, or the like. In certain embodiments, the DNA/RNA blocker is alpha-Amanitin. In certain embodiments, the DNA/RNA blocker is actinomycin D. In certain embodiments, the DNA/RNA blocker is cordycepin. In certain embodiments, the DNA/RNA blocker is fludarabine. In certain embodiments, the DNA/RNA blocker is ethidium bromide.

In certain embodiments, the method is used for treating a tumor having a FET-fused oncogene. In certain embodiments, the method is used for treating a patient (e.g., a mammal, e.g., a human) having a tumor with a FET-fused oncogene.

The present invention also features a method of treating a patient with a tumor having a FET-fused oncogene. In some embodiments, the method comprises administering to the patient an effective amount of a composition comprising a transcription inhibitor and a DNA damaging agent, wherein the composition inhibits growth of the tumor, inhibits replication of cells in the tumor, or induces cell death in cells of the tumor. Examples of such tumors include Ewing's sarcoma, desmoplastic small round cell tumor, myxoid liposarcoma, clear cell sarcoma, extraskeletal myxoid chondrosarcoma, fibromyxoid sarcoma, and those listed in Table 1. In certain embodiments, the cell having the FET-fused oncogene is a cell of a Ewing's sarcoma tumor. In certain embodiments, the cell having the FET-fused oncogene is a cell of a fibromyxoid liposarcoma tumor. In certain embodiments, the FET-fused oncogene is EWS-FLI1. In certain embodiments, the FET-fused oncogene is FUS-FLI1. In certain embodiments, the FET-fused oncogene is FUS-CHOP.

In certain embodiments, the DNA damaging agent is a topoisomerase poison. In certain embodiments, the DNA damaging agent is a DNA crosslinker. In some embodiments, the DNA damaging agent is a DNA repair inhibitor. In certain embodiments, the DNA damaging agent is an ionizing radiation therapy. In certain embodiments, the DNA damaging agent targeted radiation therapy. In certain embodiments, the DNA damaging agent is a topoisomerase poison, a DNA crosslinker, ionizing radiation therapy, or targeted radiation therapy.

For any of the embodiments herein, in certain embodiments, the topoisomerase poison may be one or a combination of is irinotecan, topotecan, camptothecin, diflomotecan, gimatecan, doxorubicin, etoposide, mitoxantrone, or daunorubicin, or the like. In certain embodiments, the topoisomerase poison is irinotecan. In certain embodiments, the topoisomerase poison is topotecan. In certain embodiments, the topoisomerase poison is camptothecin. In certain embodiments, the topoisomerase poison is diflomotecan. In certain embodiments, the topoisomerase poison is gimatecan. In certain embodiments, the topoisomerase poison is doxorubicin. In certain embodiments, the topoisomerase poison is etoposide. In certain embodiments, the topoisomerase poison is mitoxantrone. In certain embodiments, the topoisomerase poison is daunorubicin. In certain embodiments, the DNA crosslinker is one or a combination of cisplatin, carboplatin, or oxaliplatin, or the like. In certain embodiments, the DNA crosslinker is cisplatin. In certain embodiments, the DNA crosslinker is carboplatin. In certain embodiments, the DNA crosslinker is oxaliplatin. In certain embodiments, the DNA repair inhibitor is one or a combination of olaparib, veliparib, CD00509. KU-55933, vorinostat, valproic acid, or VE-821, or the like. In certain embodiments, the DNA repair inhibitor is olaparib. In certain embodiments, the DNA repair inhibitor is veliparib. In certain embodiments, the DNA repair inhibitor is CD00509. In certain embodiments, the DNA repair inhibitor is KU-55933. In certain embodiments, the DNA repair inhibitor is vorinostat. In certain embodiments, the DNA repair inhibitor is valproic acid. In certain embodiments, the DNA repair inhibitor is VE-821.

For any of the embodiments herein, in certain embodiments, the transcription inhibitor is an RNA Pol II targeting kinase inhibitor. In certain embodiments, the transcription inhibitor is a cyclin-dependent kinase inhibitor. In certain embodiments, the transcription inhibitor is a DNA/RNA blocker. In certain embodiments, the transcription inhibitor is a cyclin-dependent kinase inhibitor or a DNA/RNA blocker. In certain embodiments, the transcription inhibitor is a cyclin-dependent kinase inhibitor selected from: flavopiridol, DRB, binaciclib, roscovitine, olomoucine II, or TG02, a combination thereof, or the like. In certain embodiments, the transcription inhibitor is flavopiridol. In certain embodiments, the transcription inhibitor is DRB. In certain embodiments, the transcription inhibitor is binaciclib. In certain embodiments, the transcription inhibitor is roscovitine. In certain embodiments, the transcription inhibitor is olomoucine II. In certain embodiments, the transcription inhibitor is TG02. In certain embodiments, the DNA/RNA blocker is one or a combination of alpha-Amanitin, actinomycin D, cordycepin, fludarabine, or ethidium bromide, or the like. In certain embodiments, the DNA/RNA blocker is alpha-Amanitin. In certain embodiments, the DNA/RNA blocker is actinomycin D. In certain embodiments, the DNA/RNA blocker is cordycepin. In certain embodiments, the DNA/RNA blocker is fludarabine. In certain embodiments, the DNA/RNA blocker is ethidium bromide.

The present invention also features FET-protein fusions as markers of sensitivity to transcription inhibitors and cyclin-dependent kinase inhibitors, e.g., FET-protein fusions may be used as markers to identify and treat cancer. For example, the present invention features methods of identifying cancer using the FET-protein fusions such as but not limited to EWS-Fli1, FUS-Fli1, FUS-CHOP. For example, the presence and expression of a FET-fusion protein may be able to be confirmed by standard histological and immunohistochemical approaches with biopsy materials, as well as molecular pathology, or the like. The presence of a FET-fusion protein may be confirmed using RT-PCR, Next-Generation Sequencing methods. Northern assay, or FISH assays.

The present invention also features a composition comprising: a transcription inhibitor; and a DNA damaging agent. The composition is effective for inhibiting growth of a cell having a FET-fused oncogene, inhibiting replication of a cell having a FET-fused oncogene, or inducing cell death in a cell having a FET-fused oncogene. In certain embodiments, the composition is effective for treating a patient with a tumor having a FET-fused oncogene. As previously discussed, in certain embodiments, the DNA damaging agent is: a topoisomerase poison, a DNA crosslinker, a DNA repair inhibitor, ionizing radiation therapy, or targeted radiation therapy. In certain embodiments, the transcription inhibitor is an RNA Pol II targeting kinase inhibitor or a DNA/RNA blocker. As previously discussed, in certain embodiments, the cell having a FET-fused oncogene is a cell of a Ewing's sarcoma tumor. In certain embodiments, the cell having a FET-fused oncogene is a cell of a fibromyxoid liposarcoma tumor. In certain embodiments, the FET-fused oncogene is EWS-FLI1. In certain embodiments, the FET-fused oncogene is FUS-FLI1. In certain embodiments, the FET-fused oncogene is FUS-CHOP.

The present invention also features a composition for use in a method of inhibiting growth of, inhibiting replication of, or inducing cell death in a cell having a FET-fused oncogene, wherein the method comprises introducing to the cell having the FET-fused oncogene the composition. The composition comprises a transcription inhibitor and a DNA damaging agent. The composition is effective for inhibiting growth of the cell having a FET-fused oncogene, inhibiting replication of the cell having a FET-fused oncogene, or inducing cell death in the cell having a FET-fused oncogene. In some embodiments, the composition is effective for treating a patient (e.g., mammal, e.g., human) with a tumor having a FET-fused oncogene. As previously discussed, in certain embodiments, the DNA damaging agent is: a topoisomerase poison, a DNA crosslinker, a DNA repair inhibitor, ionizing radiation therapy, or targeted radiation therapy. In certain embodiments, the transcription inhibitor is an RNA Pol targeting kinase inhibitor or a DNA/RNA blocker. As previously discussed, in certain embodiments, the cell having a FET-fused oncogene is a cell of a Ewing's sarcoma tumor. In certain embodiments, the cell having a FET-fused oncogene is a cell of a fibromyxoid liposarcoma tumor. In certain embodiments, the FET-fused oncogene is EWS-FLI1. In certain embodiments, the FET-fused oncogene is FUS-FLI1. In certain embodiments, the FET-fused oncogene is FUS-CHOP.

For any of the embodiments herein, the presence of the FET fusion protein or the expression of the FET fusion protein may be confirmed by immunohistochemistry. For any of the embodiments herein, the presence of the FET fusion protein or the expression of the FET fusion protein may be confirmed by using an oligonucleotide-based technique, e.g., RT-PCR, FISH. northern blot, Next-Generation Sequencing (NGS), etc.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows EWS-FLI1 using multivalent properties of its low-complexity (LC) domain to assemble powerful granule bodies that interact with transcription and DNA repair machineries.

FIG. 1B shows normal interactions between FET proteins and RNA Pol II, in the context of granule assemblies, that can coordinate transcription and DNA repair pathways. Fusion proteins in Ewing sarcoma hijack this process to interrupt and destabilize both transcription regulation and DNA repair pathways.

FIG. 2A shows a comparison of the role of either EWS or EWS-FLI1 in regulating gene expression by performing RNA-seq using an Ewing's sarcoma cell line treated with knockdown or either EWS (siEWS) or EWS-FLI1 (siEWS-FLI1). By plotting the fold change of each knockdown compared to a negative control siRNA (SCR), a correlation is seen that most genes respond to either knockdown in the same manner. This is consistent with the hypothesis of both genes being cooperating in their function.

FIG. 2B shows a comparison of the results of the RNA-seq for a knockdown of EWS to that of other publicly available data for non-Ewing's cell lines. There is a noticeable increase in the ability of EWS to affect transcription with greater fold changes in Ewing's sarcoma, which has the fusion protein. This can indicate that the fusion, EWS-FLI1, has both hijacked and exaggerated EWS function in these cells. In blue and orange, respectively, are the fold changes for those genes expressed in both the Ewing's cell line (A673) and the non-Ewing's cell line (K562). In grey is shown the remainder of genes that are expressed only in the Ewing's cell line (A673*).

FIG. 2C shows a test of the functional connection between EWS and EWS-FLI1 in Ewing's sarcoma, e.g. a test of whether the knockdown of either protein would affect its ability to grow in an anchorage-independent manner, which is a parameter related to capacity for cells to metastasize. In the Ewing's cells (A673), the knockdown of EWS-FLI1 or EWS eliminated anchorage-independent colony formation. In a non-Ewing's cell line (HEK293) that expresses EWS but not the fusion, colony formation is not affected.

FIG. 2D shows quantification of four biological replicates of the colony assays shown in FIG. 2C, for SCR (i.), siEWS-FLI1 (ii.), or siEWS (iii.).

FIG. 3A shows that targeting the two major functions of FUS and EWSR1, RNA production by transcription and DNA damage repair has a synergistic effect on sarcoma cells that have a fusion protein comprised of a FET protein domain, EWS-FLI1. FUS and EWSR1 have a constitutive role in transcription and DNA damage repair. It was tested whether the functional interaction of EWS or FUS with the fusion protein driving Ewing's sarcoma would make these cells exceptionally sensitive to transcription inhibitors. A standard DNA damaging chemotherapy (SN38 or irinotecan (Stewart et al., Cell Rep 2014), used to treat Ewing's sarcoma (A673 cells) was combined with a well characterized transcription inhibitor targeting CDK9, flavopiridol. No toxicity at 96 hours was found for 1.3 nM SN38 or 100 nM flavopiridol alone. The combination treatment was highly lethal for an Ewing's sarcoma cell line (A673) but not for non-Ewing's sarcoma cells (HEK293 and U2OS). Cell survival was determined using the MTT assay. All doses shown are in nanomolar, nM.

FIG. 3B shows a western blot. During the cell's response to DNA damage, a histone protein is phosphorylated to become gammaH2Ax. It was found that the transcription inhibitor prevents this event in a Ewing's sarcoma cell line, compared to the standard DNA damage response seen when SN38 is added for 24 hours and without a transcription inhibitor. All doses shown are in nanomolar, nM.

FIG. 3C shows a western blot, wherein the experiment of FIG. 3B was performed in a non-Ewing's cell line, HEK293, the transcription inhibitor, flavopiridol, does not prevent the normal cellular response to DNA damage as indicated by the maintained levels of gammaH2Ax produced by treatment with SN38 for 24 hours. All doses shown are in nanomolar, nM.

FIG. 3D shows quantification of western blots to show the dose-dependent inhibition of the gamma H2Ax response to DNA damage in A673 cells for addition of 10 nM, 50 nM, and 100 nM flavopiridol. All doses shown are in nanomolar, nM.

FIG. 4A shows by western blot the introduction of an exogenous and recombinant EWS-FLI1 fusion protein into HEK293T cells and the removal of EWSR1 by siRNA.

FIG. 4B shows soft agar assays of non-Ewing HEK293T cells with the EWS-FLI1 fusion protein expressed. The loss of colonies upon knockdown of EWSR1 using an siEWSR1 siRNA demonstrates that the fusion protein is sufficient to recapitulate the dependency of Ewing sarcoma cells on EWSR1 expression.

FIG. 4C shows quantification of relative colony number from soft agar assays. Error bars represent standard error. Student t-test, n.s.=p>0.05. (n=6).

FIG. 4D shows western blots of pulldown assays that immunoprecipitated EWSR1 from HEK293T cells, which recovers exogenously expressed EWS-FLI1 to demonstrate that the fusion protein is sufficient to form this interaction. Mouse IgG serves as the negative control.

FIG. 5A shows EWSR1 and RNA Pol II interact with each other. Shown here is the relative levels of EWSR1 or RNA Pol II (S5P) bound and purified with each other in crosslinked granular structures (in non-Ewing cells (HEK293)).

FIG. 5B shows EWSR1 and RNA Pol interact with each other. Shown here is the relative levels of EWSR1 or RNA Pol II (S5P) bound and purified with each other in crosslinked granular structures (in Ewing cells (A673)).

FIG. 5C shows the amounts of fusion protein EWS-FLI1 bound to EWSR1 and RNA Pol 1 when the fusion is exogenously expressed in non-Ewing cells, HEK293.

FIG. 5D shows a pulldown assay revealing in Ewing sarcoma that the fusion protein, EWS-FLI1, when crosslinked in their granular structures binds both proteins.

FIG. 5E shows that mutating the fusion protein to remove its ability to make multivalent binding interactions blocks its ability to stimulate granules that recruit EWSR1 and RNA Pol to itself.

FIG. 5F shows TEM images of granules purified with RNA Pol II or with EWS-FLI1. The fusion protein exogenously expressed in a non-Ewing cell, HEK293, is sufficient to trigger granules to form.

### TERMS

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

All embodiments disclosed herein can be combined with other embodiments unless the context clearly dictates otherwise.

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons. 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

All publications, patent applications, patents, and other references mentioned herein are incorporated by reference in their entirety for all purposes. In case of conflict, the present specification, including explanations of terms, will control. Although methods and materials similar or equivalent to those described herein can be used to practice or test the disclosed technology, suitable methods and materials are described below. The materials, methods, and examples are illustrative only and not intended to be limiting.

In order to facilitate review of the various embodiments of the disclosure, the following explanations of specific terms are provided:

As used herein, the term “administration” or “administer” refers to any appropriate route of administration of a composition (e.g., pharmaceutical composition), which are well known to one of ordinary skill in the art. For example, routes of administration may include but are not limited to oral, injection (such as subcutaneous, intramuscular, intradermal, intraperitoneal, and intravenous), sublingual, rectal, ocular, otic, transdermal (e.g., topical), intranasal, vaginal and inhalation, nebulization routes, etc. The present invention is not limited to a single route of administration. For example, one compound may be administered via one particular route of administration, and a second compound may be administered via a second different route of administration. Further, administration may refer to a single dose or application, or more than one dose or application. For example, in some embodiments, the compositions herein are administered once, twice, three times, or more than three times, e.g., over a particular course or schedule.

As used herein, the term “DNA damaging agent” refers to any agent or composition that causes an abnormal chemical structure in DNA. DNA damage may impact appropriate DNA packing, DNA replication, and/or DNA transcription. Non-limiting examples of DNA damaging agents include topoisomerase poisons (e.g. irinotecan, topotecan, camptothecin, diflomotecan, gimatecan, doxorubicin, etoposide, mitoxantrone, and daunorubicin), DNA crosslinkers (e.g. cisplatin, carboplatin, and oxaliplatin), DNA repair inhibitors (e.g. olaparib, veliparib, CD00509, KU-55933, vorinostat, valproic acid, and VE-821), and ionizing or targeted radiation therapy.

As used herein, the term “disease” or “disorder” or “condition” refers to any alteration in state of the body or of some of the organs, interrupting or disturbing the performance of their functions and/or causing symptoms such as discomfort, dysfunction, distress, or even death to the person afflicted or those in contact with a person. A disease or disorder or condition can also be related to a distemper, ailing, ailment, malady, disorder, sickness, illness, complaint, indisposition or affliction.

As used herein, the term “FET-fused oncogene tumor” refers to tumors resulting from translocation events involving the FET family of factors (e.g., FUS, EWSR1, and TAF15). For example, most of Ewing's sarcomas are caused by translocations at loci containing the genes EWS and FLI1 or translocations between an EWS homologue, FUS, and FLI1. Table 1 below shows a non-limiting list of examples of fusion proteins in sarcomas. A broad range of sarcomas result from a fusion of either FUS or EWS proteins to a DNA-binding domain of a transcription factor such as FLI1. Notable among these is Ewing's Sarcoma.

TABLE 1

| Fusion Protein | Translocation | Tumour Type |
|---|---|---|
| EWSR1-FLI1 | t(11; 22)(q24; q12) | Ewing sarcoma |
| EWSR1-ERG | t(21; 22)(q22; q12) | |
| EWSR1-ETV1 | t(7; 22)(q22; q12) | |
| EWSR1-FEV | t(2; 22)(q33; q12) | |
| EWSR1-ETV4 | t(17; 22)(q12; q12) | |
| FUS-ERG | t(16; 21)(q11; q22) | |
| EWSR1-WT1 | t(11; 22)(q13; q12) | |
| EWSR1-ERG | t(21; 22)(q22; q12) | |
| FUS-DDIT3 | t(12; 16)(q13; p11) | Myxoid liposarcoma |
| EWSR1-DDIT3 | t(12; 22)(q13; p12) | |
| EWSR1-ATF1 | t(12; 22)(q13; q12) | Clear cell sarcoma |
| EWSR1-NR4A3 | t(9; 22)(q22; q12) | Extraskeletal myxoid chondrosarcoma |
| FUS-CREB3L2 | t(7; 16)(q32-34; p11) | Low-grade fibromyxoid sarcoma |
| FUS-CREB3L1 | t(7; 16)(q32-34; p11) | |
| FUS-ATF1 | t(12; 16)(q13; q11) | Angiomatoid fibrous histiocytoma |
| EWSR1-ATF1 | t(12; 22)(q13; q12) | |

As used herein, the term “Transcription Inhibitor” refers to any agent or composition that reduces or prevents transcription of a particular gene. Non-limiting examples of transcription inhibitors include RNA Pol II targeting kinase inhibitors (e.g. flavopiridol/alvocidib, DRB, binaciclib, roscovitine, olomoucine II, and TG02), and DNA/RNA blockers (e.g. alpha-Amanitin, actinomycin D, cordycepin, fludarabine, and ethidium bromide).

As used herein, the terms “treat” or “treatment” or “treating” refers to both therapeutic treatment and prophylactic or preventative measures, wherein the object is to prevent or slow the development of the disease or disorder or condition, or reducing at least one adverse effect or symptom of the condition, disease or disorder. Treatment is generally “effective” if one or more symptoms or clinical markers are reduced as that term is defined herein. Alternatively, a treatment is “effective” if the progression of a disease or disorder or condition is reduced or halted. That is, “treatment” may include not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (e.g., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with a disease or disorder or condition, as well as those likely to develop a disease or disorder or condition.

### DETAILED DESCRIPTION OF THE INVENTION

The present invention features methods and compositions for treating sarcomas such as Ewing's sarcoma. The methods and compositions of the present invention feature administering a combination of a DNA damaging agent and a transcription inhibitor.

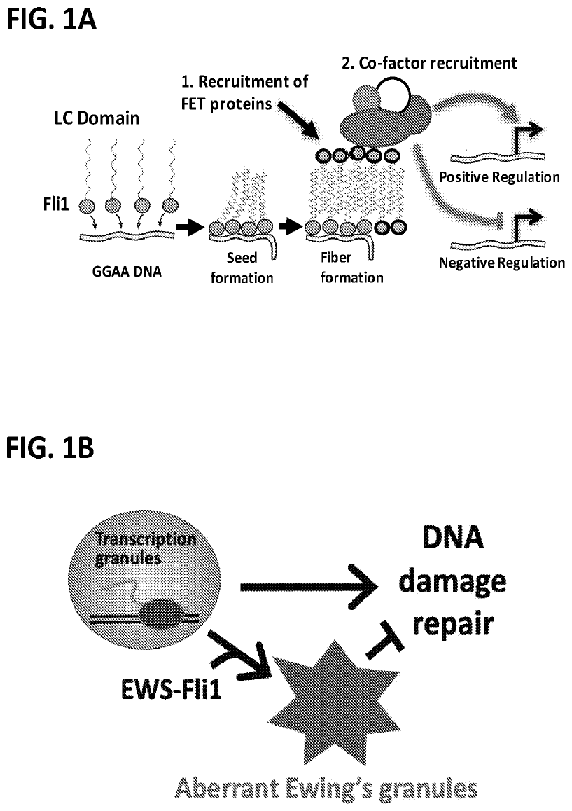

The FET protein family is comprised of FUS, EWSR1, and TAF15. These are translocated to form fusion oncogenes, including EWS-Fli1, FUS-Fli1, and FUS-CHOP, leading to several sarcomas, such as Ewing's sarcoma and fibromyxoid liposarcoma (see FIG. 1A, FIG. 1B). In fact, a fusion of either FUS or EWS to the DBD of FLI1 in the correct cell-type is sufficient for Ewing's sarcoma development. FET proteins are involved in both transcription regulation and DNA damage repair. Inventors have surprisingly found that the fusions of FET protein domains (creating oncogenes) retain some of the activities and binding partners of the original proteins, and therefore leave these tumors susceptible to therapeutic interventions that involve transcription inhibitors. Targeting both the transcription and DNA damage repair mechanisms in Ewing's sarcoma (or other appropriate conditions) provides an effective therapy.

For example, the low-complexity (LC) domains of FUS or EWS is sufficient for these to oligomerize, and these oligomers combine to form granules in cells. Without wishing to limit the present invention to any theory or mechanism, it is believed that the mechanism of action for the fusion proteins is to oligomerize with the wild-type FUS and EWS proteins to regulate the normal targets of these proteins: transcription and DNA damage repair.

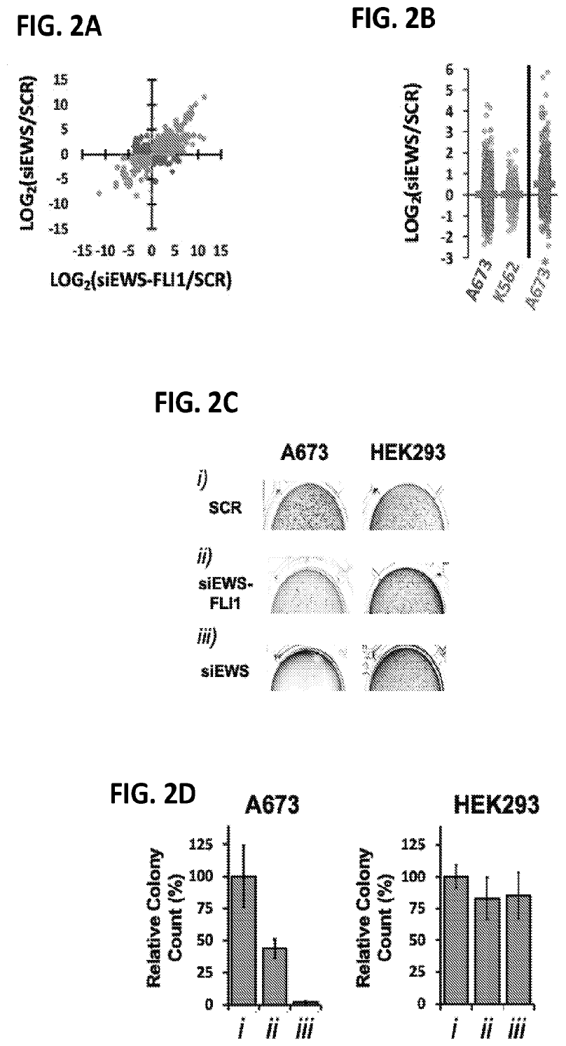

Next-generation sequencing (NGS) approaches revealed broad, complex gene expression changes triggered by FET fusion proteins. Interactions between FET and FET fusions proteins have been reported but their functional significance remains unknown. To demonstrate whether FET proteins modify fusion protein activity, a NGS study was launched to quantify RNA transcript levels (RNA-seq) in Ewing's sarcoma. siRNAs were designed to eliminate the fusion EWS-Fli1 transcript, the wildtype EWS transcript, or both: respectively, siEWS-FLI1, siEWS, and siE-EF. Knockdown was efficient in the common Ewing's sarcoma cell line, A673. RNA-seq was performed for A673 cells transfected with siEWS-FLI1 or siEWS and compared to cells transfected with a non-specific siRNA of scrambled sequence, SCR. Gene expression changes following EWS knockdown closely mirrored those for the EWS-FLI1 knockdown (see FIG. 2A). A combination of direct and downstream effects, as well as chromatin context at the FLI1 binding sites, all contribute to the observed positive and negative changes in mRNA levels. Strikingly, the magnitude of changes for both knockdowns were quite large. In the studies of FET protein activity, such large changes to mRNA levels had not been observed. Effects of EWS knockdown in A673 were compared to publicly available data in a non-Ewing cell line, K562, for those genes that are well expressed in both. It was found that the magnitude of transcript changes after EWS knockdown in the non-Ewing cells was much less than in A673 cells (see FIG. 2B).

It was tested whether EWS knockdown produced the same loss in anchorage-independent cell growth previously reported for loss of EWS-FLI1 (see FIG. 2C). Neither knockdown showed overt cell death in culture over several days. However, a complete loss of anchorage-independent cell growth was observed for EWS knockdown similar to the EWS-FLI1 knockdown (see FIG. 2C, FIG. 2D). This finding was compared to a non-Ewing cell line, HEK293. HEK293 was chosen, in part, because considerable data has been amassed for these cells and they are particularly amenable to a broad range of molecular techniques modifying genes and gene expression. As expected, siEWS-FLI1 had no effect on colony formation in HEK293 (see FIG. 2C, FIG. 2D). Similarly, knockdown of EWS did not eliminate anchorage-dependent cell growth.

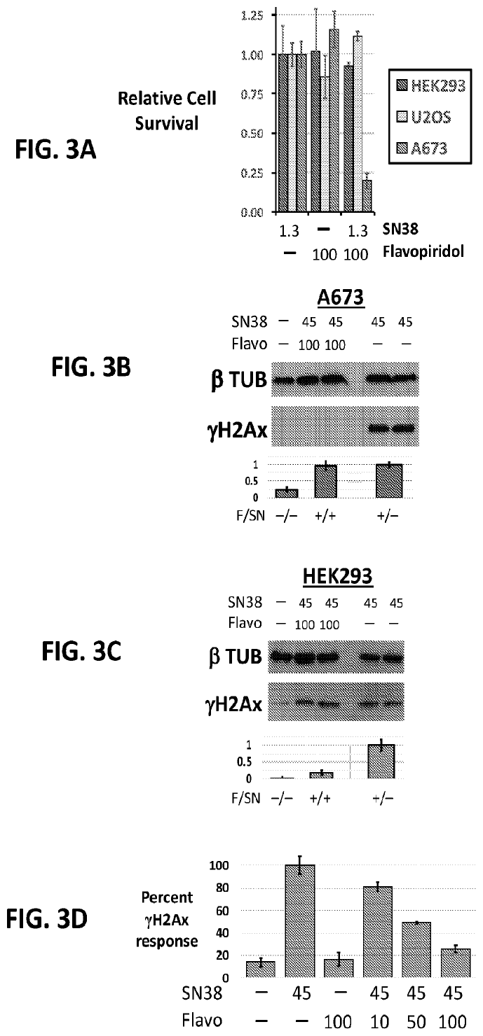

The DNA damage agent SN38 and the transcription inhibitor flavopiridol were titrated, and cell survival was measured after 96 hours using an MTT assay (see FIG. 3A). In the Ewing's sarcoma cell line, A673, the combined treatment induced significant cell death but not for non-Ewing cell lines HEK293 or U2OS. In agreement with this finding, confirmation by western of the DNA damage cellular response of phosphorylating a histone to produce gammaH2Ax, revealed that addition of the transcription inhibitor, flavopiridol, prevented this event in Ewing's sarcoma (see FIG. 3B) but had no such effect on a non-Ewing's cell line, HEK293 (see FIG. 3C).

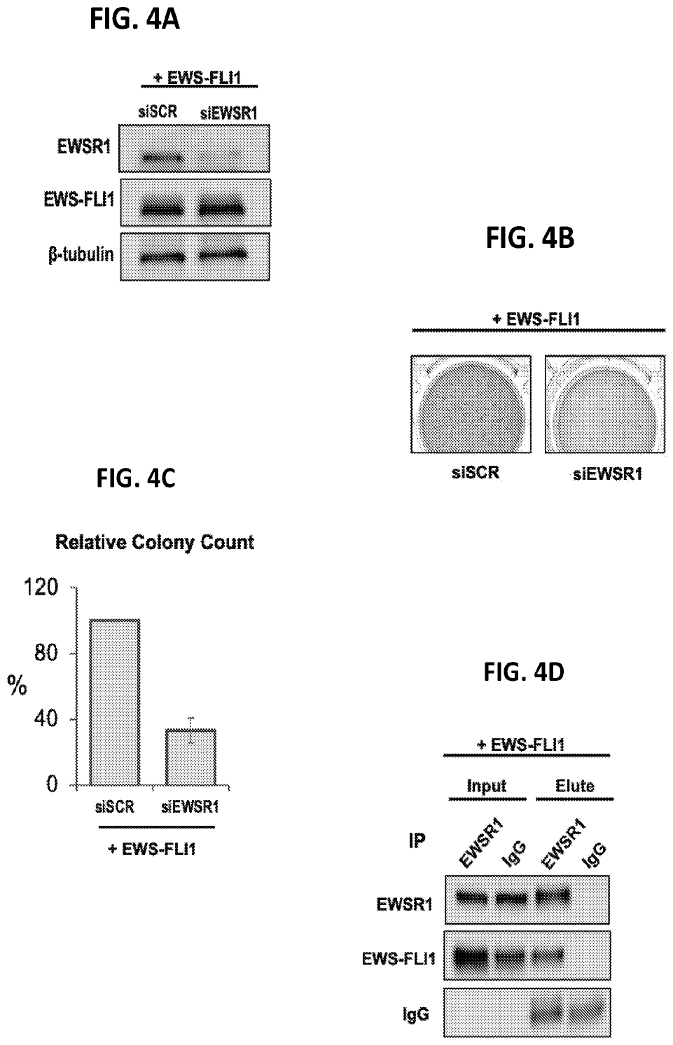

Referring to FIG. 4A, FIG. 4B, FIG. 4C, and FIG. 4D, to confirm the model that targeting both FET protein functions simultaneously would produce a more effective treatment approach, a physical interaction between the FET protein, EWS, and the fusion protein EWS-FLI1 was confirmed. By expressing a tagged fusion protein EWS-FLI1 in a non-Ewing's cell line, HEK293T cells, a pulldown assay for EWS also recovers the fusion protein EWS-FLI1. In the converse experiment, a pulldown of EWS-FLI1 recovers EWS. Similarly, a pulldown of the RNA polymerase responsible for mRNA production in all human cells, RNA Pol 1, recovered EWS in a non-Ewing's cell line, HEK293, and both EWS and the fusion, EWS-FLI1, in an Ewing's cell line.

It will be appreciated that appropriate dosages of the compositions herein may vary from patient to patient. Further, the selected dosage level may depend on a variety of factors including, but not limited to: the activity of the particular composition, the route of administration, the time of administration, the rate of excretion or metabolism of the composition (e.g., the drug portion), the duration of the treatment, other drugs, compounds, and/or materials used in combination, and the age, sex, weight, condition, general health, and prior medical history of the patient. The amount of compound and route of administration may be at the discretion of the physician, although generally the dosage will be to achieve local concentrations at the site of action that achieve the desired effect without causing substantial harmful or deleterious side-effects.

In certain embodiments, administration in vivo can be achieved in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out.

The compositions herein may be appropriately constructed for some or all routes of administration, e.g., oral or enteral administration, intravenous or parenteral administration, topical administration (including inhalation and nasal administration), transdermal administration, epidural administration, and/or the like. For example, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, compositions to be administered can contain minor amounts of non-toxic auxiliary substances, such as wetting or emulsifying agents, preservatives, and pH buffering agents and the like, for example sodium acetate or sorbitan monolaurate.

Table 2 below lists non-limiting examples of compositions, wherein the compositions comprise a transcription inhibitor and a DNA damaging agent.

| Example | Transcription Inhibitor | DNA Damaging Agent |
|---|---|---|
| 1 | Cyclin-dependent kinase inhibitor | topoisomerase poison |
| 2 | Cyclin-dependent kinase inhibitor | DNA crosslinker |
| 3 | Cyclin-dependent kinase inhibitor | ionizing radiation therapy |
| 4 | Cyclin-dependent kinase inhibitor | DNA repair inhibitor |
| 5 | Cyclin-dependent kinase inhibitor | radiation therapy |
| 6 | DNA/RNA blocker | topoisomerase poison |
| 7 | DNA/RNA blocker | DNA crosslinker |
| 8 | DNA/RNA blocker | ionizing radiation therapy |
| 9 | DNA/RNA blocker | DNA repair inhibitor |
| 10 | DNA/RNA blocker | radiation therapy |
| 11 | flavopiridol, DRB, binaciclib, roscovitine, | irinotecan, topotecan, camptothecin, |
| | olomoucine II, or TG02 | diflomotecan, gimatecan, doxorubicin, |
| | | etoposide, mitoxantrone, or daunorubicin |
| 12 | alpha-Amanitin, actinomycin D, cordycepin, | irinotecan, topotecan, camptothecin, |
| | fludarabine, or ethidium bromide | diflomotecan, gimatecan, doxorubicin, |
| | | etoposide, mitoxantrone, or daunorubicin |
| 13 | flavopiridol, DRB, binaciclib, roscovitine, | cisplatin, carboplatin, or oxaliplatin |
| | olomoucine II, or TG02 | |
| 14 | alpha-Amanitin, actinomycin D, cordycepin, | cisplatin, carboplatin, or oxaliplatin |
| | fludarabine, or ethidium bromide | |
| 15 | flavopiridol, DRB, binaciclib, roscovitine, | olaparib, veliparib, CD00509, KU-55933, |
| | olomoucine II, or TG02 | vorinostat, valproic acid, or VE-821 |
| 16 | alpha-Amanitin, actinomycin D, cordycepin, | olaparib, veliparib, CD00509, KU-55933, |
| | fludarabine, or ethidium bromide | vorinostat, valproic acid, or VE-821 |
| 17 | flavopiridol, DRB, binaciclib, roscovitine, | Ionizing radiation |
| | olomoucine II, or TG02 | |
| 18 | alpha-Amanitin, actinomycin D, cordycepin, | Ionizing radiation |
| | fludarabine, or ethidium bromide | |
| 19 | flavopiridol, DRB, binaciclib, roscovitine, | Radiation therapy |
| | olomoucine II, or TG02 | |
| 20 | alpha-Amanitin, actinomycin D, cordycepin, | Radiation therapy |
| | fludarabine, or ethidium bromide | |
| 21 | flavopiridol | irinotecan |
| 22 | flavopiridol | topotecan |
| 23 | flavopiridol | cisplatin |
| 24 | flavopiridol | olaparib |
| 25 | flavopiridol | Radiation therapy |
| 26 | TG02 | irinotecan |
| 27 | TG02 | topotecan |
| 28 | TG02 | cisplatin |
| 29 | TG02 | olaparib |
| 30 | TG02 | Radiation therapy |
| 31 | alpha-Amanitin | irinotecan |
| 32 | alpha-Amanitin | topotecan |
| 33 | alpha-Amanitin | cisplatin |
| 34 | alpha-Amanitin | olaparib |
| 35 | alpha-Amanitin | Radiation therapy |

As previously discussed, the presence of a FET-fused oncogene gives rise to a synergistic effect between inhibition of transcription and DNA damage. Under these conditions, the addition of a transcription inhibitor can lead to greater effectiveness for DNA damage treatments. Similarly, the FUS-fused protein will allow transcription inhibitors to provide a therapeutic effect at doses far lower when combined with the DNA damage treatment than alone.

The method of leveraging the synergy between transcription inhibitors and DNA damage agents in tumors having the FET-fusion protein can allow a therapeutic effect at lower doses or subdose of the agents alone. The presence of the FET-fusion protein can be used as a biomarker of tumors that can be exceptionally sensitive to a combination treatment using transcription inhibiting agents and DNA damaging therapies.

Therapeutic doses of transcription inhibitors (such as the CDK and transcription inhibitor flavopiridol) has shown significant toxicity in patients, which has prevented its clinical use for most tumors to enter trials to date. Combination treatments of DNA damage treatments and the maximum tolerated dose flavopiridol can enhance therapeutic benefits but as yet has failed to reach clinical significance (Ang et al., Gastrointest Cancer Res 2012; Cicenas et al., Cancers (Basel) 2014). Illustrating the surprising findings of the synergistic effect between inhibition of transcription and DNA damage, Inventors have found that tumor cells possessing the FET-fusion protein cannot grow or survive at doses of flavopiridol and the DNA damage agent irinotecan three to ten times (3-10 times) lower than those doses that have been used to achieve therapeutic effect in clinical trials (Ducreux et al., Ann Oncol 2003; Shah et al., Clin Cancer Res 2005; Deep et al., New J Chem 2018).

Doses used in the present invention, e.g., of the transcription inhibitor and/or DNA damaging agent, may be sub-efficacious, sub-clinical, low dose, etc., e.g., doses that may be lower than what is typically used in clinical (or in vitro) settings. For example, doses of flavopiridol used to stop cells from growing are in the 200 to 500 nm range, and these are the same plasma levels sought for therapeutic effects in trials. In the present invention, doses used in cells having the fusion protein is from about 50 to 100 nM. However, the present invention is not limited to the aforementioned doses.

### Example 1

The following are non-limiting examples of methods used for the present invention. It is to be understood that said example is not intended to limit the present invention in any way. Equivalents or substitutes are within the scope of the present invention.

Cell lines were obtained from ATCC. A673 and SK-N-MC cells were grown in DMEM supplemented with 10% FBS. HEK293T/17 cells were grown in DMEM supplemented with 5% FBS. All cell lines were cultured at 37° C. and 5% CO₂.

siRNA transfections: A673 cells were reverse transfected at 5.0×10⁵ cells in 6-well dishes using RNAiMAX (Life Technologies). HEK293 and SK-N-MC cells were plated at 4.0×10⁵ in 6-well dishes 24 hours prior to transfection using the TransIT-X2 reagent (Mirus Bio cat #MIR6000) or RNAIMAX (Life Technologies). All siRNA transfections were at a final concentration of 50 nM and cells were harvested 48 hours post-transfection for analysis by western blot.

plasmid transfections: HEK293T/17 cells were transfected at 80% confluency. Cells were transfected using TransIT-X2 reagent (Mirus Bio) following manufacturer's instructions. Cells were harvested 48-96 hr hours post-transfection.

Co-Immunoprecipitation assay: Cells were harvested from 6-well plates and lysed in ColP lysis/wash buffer (25 mM Tris-HCl pH 7.4, 200 mM NaCl, 1 mM EDTA, 0.5% NP40, 5% glycerol). Protein A/G agarose beads (EMD Millipore) were incubated with primary antibody for 2 hours at 4° C. before addition to cell lysate. Lysate was incubated with beads-antibody complex overnight with rotation at 4° C. Beads were washed five times in ColP lysis/wash buffer, resuspended in Novex NuPage Sample Loading Buffer (Fischer Scientific) with 5 mM DTT, and boiled for 5 min at 95° C. Beads were then spun at 8000 rpm to elute protein complexes. ColP assays were then assessed by western blot.

Crosslinked immunoprecipitation assay: Cells were harvested from confluent 150 mm dishes. Cells were crosslinked in 1% formaldehyde for 15 minutes and then quenched in 125 mM glycine. Cells were washed in PBS, lysed in Buffer B (1% SDS, 10 mM EDTA, 50 mM Tris-HCl pH 8.0) supplemented with protease inhibitors and sonicated using the Bioruptor Pico (Diagenode) for 30 minutes, and then spun at max speed for 30 minutes at 4° C. Crosslinked lysate was diluted 10-fold in IP lysis buffer (0.01% SDS, 1.1% Triton-X, 1.2 mM EDTA, 16.7 mM Tris-HCl pH 8.0, 167 mM NaCl) treated with protease inhibitors (Sigma-Aldrich) and benzonase (Millepore-Sigma), and incubated with rotation overnight with primary antibody at 4° C. Crosslinked granules were immunoprecipitated with Novex DYNAL Dynabeads Protein G (Invitrogen) or Protein A/G agarose beads (EMD Millipore) for 2 hours at room temperature. Beads were washed five times using IP lysis buffer and eluted in 3.6 M MgCl₂ and 20 mM MES (pH 6.5) for 30 minutes with agitation. IP samples were then assayed for proteins by ELISA.

ELISA: Crosslinked immunoprecipitation samples or SEC fractions were plated onto a Greiner LUMITRAC-600 plate (VWR). Samples were incubated overnight at 4° C. Samples were washed 3 times in TBST, blocked for 2 hours at room temperature in 5% nonfat dried milk in 0.1% TBST, washed 4 times in TBST and then incubated with primary antibody overnight at 4° C. or for 2 hours at room temperature. Samples were then washed 4 times again in TBST and incubated with secondary antibody for 1 hour at room temperature before washing 4 times with TBST. SuperSignal™ ELISA Femto substrate (Thermo Scientific) was used to detect protein and the plate was read on the

Western blot analysis: Protein lysates concentrations were quantified using BCA Protein Assay (Pierce). 5-10 ug of protein sample was loaded onto 7.5% SDS-PAGE gels. Standard western blot protocols were followed. Blots were transferred at 500 mA, blocked in 5% nonfat dried milk in TBS-T, and incubated overnight with primary antibody at 4° C. Blots were washed in TBS-T, incubated in secondary antibody for 1 hour at room temperature, washed again in TBS-T, and imaged after the addition of SuperSignal™ West Pico PLUS Chemiluminescent substrate (Thermo Scientific). Western blots were imaged on the ChemiDoc.

Soft agar colony formation assay: Soft agar assays were performed using 1.2% Nobel Agar and DMEM supplemented with FBS and penicillin/streptomycin. The underlayer was made of 0.6% agar. A673 cells transfected with 50 nM siRNA were collected 24 hours post-transfection. HEK293T/17 cells transfected with 50 nm siRNA or 2 ug of plasmid DNA were collected 24 hours post-transfection. A673 and SK-N-MC cells were seeded at a density 1.0×10⁵ cells in 0.35% agar and HEK293T/17 cell were seeded at 2.0 to 3.0×10⁴ in 0.4% agar in media similar to the composition described above, which was then plated over the agar underlayer. A673 and SK-N-MC cells were grown at 37 C and 5% CO2 for 3-4 weeks, imaged, and then colonies were counted using ImageJ software. HEK293T/17 were grown at 37 C and 5% CO2 for 1-2 weeks, imaged, and then colonies were counted. Colonies with stained with 0.005% methylene blue.

RNA-sequencing: A673 cells were transfected with 50 nM siRNA as described above. Cells were collected 72 hours post-transfection and total RNA was extracted using TRIzol reagent (Thermo Fischer). 1 ug of total RNA was prepared for sequencing using NEBNext Poly(A) mRNA Magnetic Isolation Module (NEB) to generate sequencing libraries according to manufacturer's instructions.

### Example 2

The following is a non-limiting of the present invention. It is to be understood that said example is not intended to limit the present invention in any way. Equivalents or substitutes are within the scope of the present invention.

The low-complexity domain of FET proteins has the propensity to phase separate and form protein granules in cells. It is also known to bind to the C-terminal domain of RNA Pol II to regulate transcription. It has been shown that FUS, a close homologue of EWSR1 and also another translocation partner of FLI1 in Ewing sarcoma, exists with RNA Pol II in a granule together. However, it has not been determined if EWSR1 is found in these granules in cells.

The present invention describes a method combining formaldehyde crosslinking and size-exclusion chromatography (SEC). Formaldehyde is a close-distance crosslinker that covalently bonds proteins within single-angstrom distances and stabilizes protein granules. From SEC, the protein granules elute first from the column due to their large size, while smaller complexes and protein monomers elute towards the end of the column. HEK293 cells were crosslinked with 1% formaldehyde to stabilize granule particles, lysed in 6M urea, and ran over a CL-2B column. Uncrosslinked HEK293 cells were also ran over the CL-2B column as a negative control. The UV trace of total protein shows the majority of proteins eluted in the monomer form at an elution volume of around 20 ml (see FIG. 4A). The SEC fractions were tested for RNA Pol II and EWSR1 by ELISA. RNA Pol II eluted at the beginning of the column at around 10 ml, which resolves particles at 50-100 nm in size, a size much larger than the RNA Pol II holoenzyme itself (see FIG. 4B). There were also peaks for RNA Pol II eluting in later fractions, suggesting not all of the polymerase was in a large protein granule but also in smaller complexes. Strikingly, almost all of EWSR1 also eluted at the beginning of the column in large particles. EWSR1 eluted in the same fractions as RNA Pol II, suggesting that EWSR1 may be in large granules along with RNA Pol II.

In additional crosslinked immunoprecipitation assays, HEK293T cells were crosslinked using 1% formaldehyde. lysed and sonicated, and the lysate was used immunoprecipitate EWSR1 and RNA Pol II. Significant enrichment of RNA Pol II with EWSR1 was found using an antibody that detects the serine-5 phosphorylated form of RNA Pol II (see FIG. 4D). This indicates that EWSR1 and RNA Pol II exist in large granules together in HEK293 cells. Crosslinked immunoprecipitation assays were also performed in A673 cells and the same enrichment trend of EWSR1 and RNA Pol II together in HEK293 cells was observed (see FIG. 4E). This suggests granules of EWSR1 interacting with RNA Pol II is a ubiquitous function of EWSR1. Furthermore, the enrichment of EWSR1 with serine-5 phosphorylated form of RNA Pol II suggests these granules are transcriptionally active.

The shared low-complexity domain of EWSR1 and EWS-FLI1 implies that EWS-FLI1 may have the same phase-separation properties of EWSR1. Indeed, EWS-FLI1 has been shown to be capable of phase-separating and forms nuclear puncta, suggesting the formation of granular bodies.

Using a protocol to crosslink cells to stabilize protein granules, the crosslinking immunoprecipitation assay was performed to immunoprecipitate granules and assess for the presence of EWSR1 and EWS-FLI1. Enrichment of EWS-FLI1 was found when crosslinked EWSR1 was immunoprecipitated from HEK293 cells transfected with EWS-FLI1. EWS-FLI1 was also found with RNA Pol II granules. Furthermore, to confirm that these granule interactions are maintained in Ewing sarcoma cells, EWSR1 and RNA Pol II was immunoprecipitated from crosslinked A673 cells. It was not determined if EWS-FLI1 was also enriched in EWSR1 and RNA Pol II granules.

Mutations of tyrosines in the low-complexity to serine residues has been shown to disrupt phase-separation capacity in vitro. Additionally, as the low-complexity domain mediates interactions with protein partners, Y-to-S mutations also inhibits protein-protein interactions. A mutant of EWS-FLI1 in which the 37 tyrosines of the low-complexity domain were mutated to serine residues (EWS(YS37)-FLI1) was transfected into HEK293 cells. Strikingly, significant enrichment of EWS-FLI1 was observed over the control when EWSR1 and RNA Pol II was immunoprecipitated from HEK293 cells transfected with the Y-to-S EWS-FLI1 mutant. The same effect was observed when the EWS(YS37)-FLI1 mutant was immunoprecipitated.

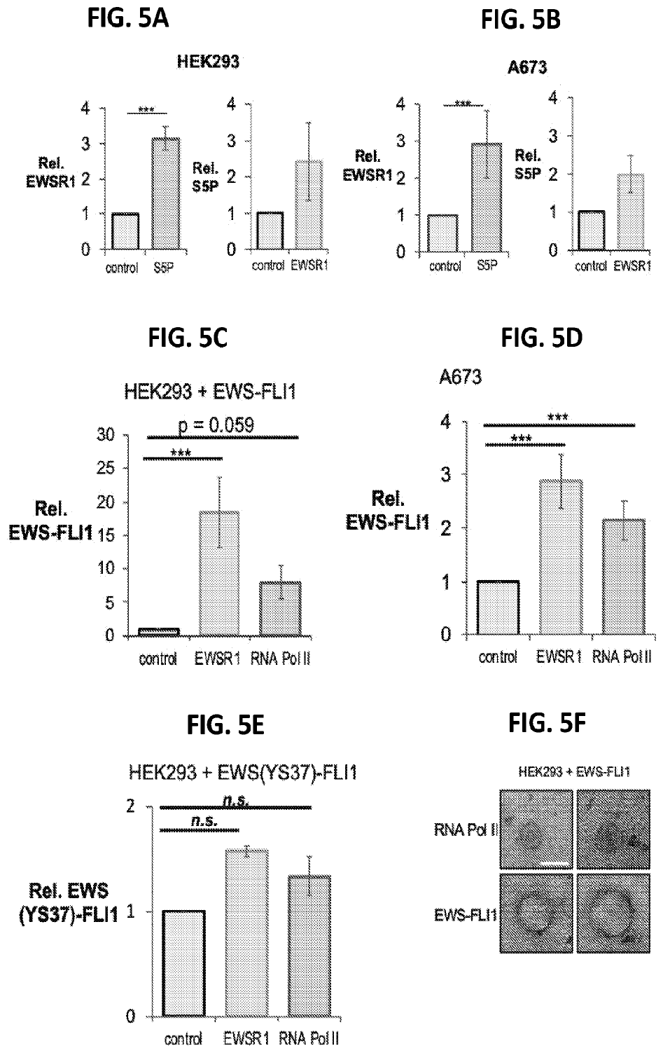

Referring to FIG. 5A, FIG. 5B, FIG. 5C, FIG. 5D, FIG. 5E, and FIG. 5F, while nuclear foci of EWS-FLI1 can be observed in cells, crosslinked immunoprecipitation samples were spotted onto carbon grids and stained with uranyl formate and imaged by transmission electron microscopy. RNA Pol II granules were identified that were 30-35 nm in size (see FIG. 5A). As the holoenzyme of RNA Pol II is estimated to be 15-25 nm, this suggests that RNA Pol II was in granules larger than the size of the polymerase alone. Furthermore, these particles were observed were round and circular in shape, corresponding to in-vitro experiments of recombinant proteins condensates have been shown form large spherical shapes. Additionally, treatment of crosslinked immunoprecipitation samples with proteinase K resulted in loss of circular particles, indicating that these particles are indeed comprised of protein and may accurately represent protein granules in cells.

Immunoprecipitation samples of EWS-FLI1 from HEK293 transfected with the fusion protein were imaged. Similar to the RNA Pol II granules, large circular EWS-FLI1 particles were observed. The EWS-FLI1 granules appeared in the size range of 60-80 nm, which is much larger than a single EWS-FLI1 monomer. As EWS-FLI1 multimerizes on the chromatin at GGAA microsatellites and recruits and interacts with large gene regulation machinery, such as RNA Pol II, the size of these particles indicate they are indeed granules of EWS-FLI1. Smaller circular particles of EWS-FLI1 that ranged in size from 10-15 nm were also observed. It was hypothesized that these may be “seeds” of EWS-FLI1. In contrast to the EWS-FLI1 granules, similar morphology of particles of the Y-to-S mutant of EWS-FLI1 was not observed. This would suggest the Y-to-S mutation abrogates the ability of EWS-FLI1 to form granules in cells.

The present invention describes a novel mechanism through which EWSR1 may affect the oncogenic activity of EWS-FLI1. While EWSR1 has been linked to several cellular processes, the phase-transition properties of EWSR1 has not been tied its function in the cell. It was established that EWSR1 exists in large granules in cells by size-exclusion chromatography. Furthermore, it was possible to identify protein partners associated with EWSR1 in these assemblies. It was shown that part of the ubiquitous function of EWSR1 is interacting with RNA Pol II in large granules. The presence of EWS-FLI1 did not after the enrichment of EWSR1 with RNA Pol II, as EWS-FLI1 itself was also found in EWSR1 and RNA Pol II granules. Most notably, these granules were resolved by transmission electron microscopy. Previous studies have shown that components of the BGR1/BRM-associated factor (BAF) chromatin remodeling complex are another partner of EWSR1 in cells. EWS-FLI1 also interacts with the BAF complex in Ewing sarcoma cells. This, coupled with the present invention, suggests that EWS-FLI1 may conscript wild-type functions of EWSR1, such as its protein partners, which may after transcriptional output.

In conjunction with EWS-FLI1 conscripting EWSR1 function, it was observed that EWSR1 and EWS-FLI1 share the same phenotypic outputs, such as inhibiting anchorage-independent growth upon loss of expression in Ewing sarcoma cells. Surprisingly, this effect is not observed upon loss of EWSR1 in a non-Ewing sarcoma cell line, but is recapitulated upon expression of EWS-FLI1. It has been shown that EWSR1 and EWS-FLI1 interact by their low-complexity domain in Ewing sarcoma cells. It was found that this interaction persists even when EWS-FLI1 is exogenously expressed in HEK293T cells and is important for the transformation properties of EWS-FLI1. In addition, it was found that EWSR1 and EWS-FLI1 share a similar molecular phenotype, where they closely regulate the same genes in a similar manner. This would suggest that EWSR1 and EWS-FLI1 may maintain a similar oncogenic program in Ewing sarcoma cells.

Low-complexity domains are intimately tied to normal cell processes and disease states. Ewing sarcoma presents a unique model where wild-type and aberrant low-complexity domain proteins exist in the same system. Without wishing to limit the present invention to any theory or mechanism, it is hypothesized that EWS-FLI1 may conscript ubiquitous EWSR1 function. This falls in line with studies showing how the close EWSR1 homologue, FUS, is also normally found in large granules with RNA Pol 1. These granules are transcription-driven, such that treatment with transcription-inhibiting drugs causes RNA Pol to come out of granules. This would imply wide-scale transcriptional changes. As EWSR1 and EWS-FLI1 also form granules with RNA Pol II, identifying the molecular and chemical determinants that maintain these assemblies would offer a novel therapeutic avenue by which to disassemble oncogenic transcriptional output.

FET proteins and their low-complexity domains are frequent partners of translocation events in sarcomas. This highlights importance of understanding how low-complexity domain driven phase-separated granules may be conscripted in a cancer setting.

### Example 3

The following is a non-limiting of the present invention. It is to be understood that said example is not intended to limit the present invention in any way. Equivalents or substitutes are within the scope of the present invention.

A patient presents to a clinical trial having been diagnosed with Ewing Sarcoma. The physicians of the clinical trial administer the patient: (1) a dose of flavopiridol that achieves a serum level of 100 nM: in combination with (2) a 50 m/m² dose of irinotecan for 4 weeks. Following the 4 week treatment, the patient shows a reduction in significant tumor volume. The patient is administered a second treatment using the same doses of flavopiritol and irinotecan for an additional 4 weeks. Following the second treatment, the patient shows another significant reduction in tumor volume.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

1. A method of inhibiting growth of, inhibiting replication of, or inducing cell death in a cell having a FET-fused oncogene, said method comprising introducing to the cell having the FET-fused oncogene an effective amount of a composition comprising a transcription inhibitor and a DNA damaging agent, wherein the composition inhibits growth of the cell, inhibits replication of the cell, or induces cell death in the cell.

2. The method of claim 1, wherein the cell having the FET-fused oncogene is a cell of a Ewing's sarcoma tumor or a fibromyxoid liposarcoma tumor.

3. (canceled)

4. The method of claim 1, wherein the FET-fused oncogene is EWS-FLI1, FUS-FLI1 or FUS-CHOP.

5.-6. (canceled)

7. The method of claim 1, wherein the DNA damaging agent is: a topoisomerase poison, a DNA crosslinker, DNA repair inhibitor, ionizing radiation therapy, or targeted radiation therapy.

8. The method of claim 7, wherein the topoisomerase poison is irinotecan, topotecan, camptothecin, diflomotecan, gimatecan, doxorubicin, etoposide, mitoxantrone, or daunorubicin.

9. The method of claim 7, wherein the DNA crosslinker is cisplatin, carboplatin, or oxaliplatin.

10. The method of claim 7, wherein the DNA repair inhibitor is olaparib, veliparib, CD00509, KU-55933, vorinostat, valproic acid, or VE-821.

11. The method of claim 1, wherein the transcription inhibitor is an RNA Pol II targeting kinase inhibitor or a DNA/RNA blocker.

12. The method of claim 11, wherein the transcription inhibitor is a cyclin-dependent kinase inhibitor selected from: flavopiridol, DRB, binacidib, roscovitine, olomoucine II, or TG02.

13. The method of claim 11, wherein the DNA/RNA blocker is alpha-Amanitin, actinomycin D, cordycepin, fludarabine, or ethidium bromide.

14. The method of claim 1, wherein the method is used for treating a tumor having a FET-fused oncogene.

15. The method of claim 1, wherein the method is used for treating a patient having a tumor with a FET-fused oncogene.

16. The method of claim 1, wherein presence of the FET fusion protein or expression of the FET fusion protein is confirmed by immunohistochemistry or an oligonucleotide-based technique.

17. (canceled)

18. The method of claim 16, wherein the oligonucleotide-based technique is RT-PCR, FISH, northern blot, or Next-Generation Sequencing (NGS).

19. A method of treating a patient with a tumor having a FET-fused oncogene, said method comprising: administering to the patient an effective amount of a composition comprising both a transcription inhibitor and a DNA damaging agent, wherein the composition inhibits growth of the tumor, inhibits replication of cells in the tumor, or induces cell death in cells of the tumor.

20. The method of claim 19, wherein the tumor having a FET-fused oncogene is a Ewing's sarcoma tumor or a fibromyxoid liposarcoma tumor.

21. (canceled)

22. The method of claim 19, wherein the FET-fused oncogene is EWS-FLI1 or FUS-FLI1, or FUS-CHOP.

23.-24. (canceled)

25. The method of claim 19, wherein the DNA damaging agent is a topoisomerase poison, a DNA crosslinker, DNA repair inhibitor, Ionizing radiation therapy, or targeted radiation therapy.

26.-28. (canceled)

29. The method of claim 19, wherein the transcription inhibitor is an RNA Pol II targeting kinase inhibitor or a DNA/RNA blocker.

30.-32. (canceled)

33. The method of any of claim 19, wherein the patient is a human.

34-62. (canceled)
